(19) United States
(12) Patent Application Publication
(10) Pub. No.: US 2003/0082624 A1
(43) Pub. Date: May 1, 2003

(54) Method and system to investigate a complex chemical space

(75) Inventor: James Norman Cawse, Pittsfield, MA (US)

Correspondence Address: GENERAL ELECTRIC COMPANY, GLOBAL RESEARCH CENTER, PATENT DOCKET RM. 4A59, PO BOX 8, BLDG. K-1 ROSS, NISKAYUNA, NY, 12309, US

(73) Assignee: General Electric Company

(21) Appl. No.: 09/938,763

(22) Filed: Aug. 27, 2001

(51) Int. Cl. G01N 33/53; G06G 7/48; G06G 7/58; G06F 19/00; G01N 33/48

(52) U.S. Cl. 435/7.1; 702/19; 436/518

(57) ABSTRACT

An experimental space of a catalyzed chemical reaction is defined to represent at least three factor interactions, a CHTS method is effected on the catalyzed chemical experimental space to produce results and results are analyzed by matrix algebra to select a best case set of factor levels from the catalyzed experimental space. A system for investigating a catalyzed experimental space comprises a reactor for effecting a CHTS method on the catalyzed chemical experimental space to produce results and a programmed controller to analyze the results by matrix algebra to select a best case set of factor levels from the catalyzed experimental space.

This invention was made with government support under Contract No. 70NANB9H3038 awarded by NIST. The government may have certain rights to the invention.

### BACKGROUND OF THE INVENTION

The present invention relates to a combinatorial high throughput screening method and system.

Combinatorial organic synthesis (COS) is a high throughput screening method that was developed for pharmaceuticals. COS uses systematic and repetitive synthesis to produce diverse molecular entities formed from sets of chemical “building blocks.” As with traditional research, COS relies on experimental synthesis methodology. However instead of synthesizing a single compound, COS exploits automation and miniaturization to produce large libraries of compounds through successive stages, each of which produces a chemical modification of an existing molecule of a preceding stage. Libraries are physical, trackable collections of samples resulting from a definable set of the COS process or reaction steps. The libraries comprise compounds that can be screened for various activities.

Combinatorial high throughput screening (CHTS) is an HTS method that incorporates characteristics of COS. The CHTS methodology is marked by the search for high order synergies and effects of complex combinations of experimental variables through the use of large arrays in which multiple factors can be varied through multiple levels. Factors of an experiment can be varied within an array (typically formulation variables) and between an array and a condition (both formulation and processing variables). Results from the CHTS experiment can be used to compare properties of the products in order to discover “leads”—formulations and/or processing conditions that indicate commercial potential.

The steps of a CHTS methodology can be broken down into generic operations including selecting chemicals to be used in an experiment; introducing the chemicals into a formulation system (typically by weighing and dissolving to form stock solutions), combining aliquots of the solutions into formulations or mixtures in a geometrical array (typically by the use of a pipetting robot); processing the array of chemical combinations into products; and evaluating the products to produce results.

Typically, CHTS methodology is characterized by parallel reactions at a micro scale. In one aspect, CHTS can be described as a method comprising (A) an iteration of steps of (i) selecting a set of reactants; (ii) reacting the set and (iii) evaluating a set of products of the reacting step and (B) repeating the iteration of steps (i), (ii) and (iii) wherein a successive set of reactants selected for a step (i) is chosen as a result of an evaluating step (iii) of a preceding iteration.

Results from an experiment can be evaluated by aid of mathematical models as taught by G.E.P. Box and N. R. Draper, Empirical Model-Building and Response Surfaces, John Wiley and Sons, NY, 1987, p 20-22. The models can be used to find an approximation, typically a polynomial, to an unknown underlying theoretical function. For example, Taylor's Series expansion is a polynomial that can provide a valuable approximation of first or second order experimental interactions.

However, the study of catalyzed chemical reactions by CHTS involves the investigation of a complex experimental space characterized by multiple qualitative and quantitative factor levels. Typically, the interactions of a catalyzed chemical reaction such as a carbonylation reaction can involve interactions of an order of 6 or 9 or greater. While Taylor expansion approximation can be effectively applied to analyze first or second order interactions, it is useless to study CHTS results from a complex catalyzed chemical reaction.

Another problem is that catalyzed chemical reactions can be unpredictable. Well-known protocols in one area of chemistry cannot be applied to another area with assurance of success. For example, U.S. Pat. No. 6,143,914 shows that some combinations of various metals unexpectedly increase a carbonylation catalyst turnover number (TON) and other related combinations do not. “Due to the complicated mechanistic nature of many transition metal based catalysts, structure-activity relationships are often unpredictable, leaving empirical exploration and serendipity the most common routes to discovery.” J. Tian & G. W. Coates, Angew. Chem Int. Ed. 2000, 39, p 3626. This high degree of irregularity and unpredictability is illustrated in FIG. 1.

There is a need for a methodology to examine the complex higher order and unpredictable interactions of a CHTS catalyzed chemical reaction experiment that cannot be examined by Taylor Series expansion or other standard methodology.

### BRIEF SUMMARY OF THE INVENTION

The invention provides a particularly well-suited experimental methodology to investigate multiple and complex interactions of a catalyzed chemical reaction that involves both qualitative and quantitative factor levels. According to the invention, an experimental space of a catalyzed chemical reaction is defined to represent at least three factor interactions, a CHTS method is effected on the catalyzed chemical experimental space to produce results and results are analyzed according to matrix algebra to select a best case set of factor levels from the catalyzed experimental space.

In another embodiment, a CHTS experiment is conducted on a complex experimental space comprising qualitative and quantitative factors to produce first data results, (B) the first data results are analyzed according to matrix algebra, (C) a standard deviation of the analyzed results is defined, (D) data results that positively exceed the standard deviation are selected, (E) a next experimental space is defined according to the selected data results and (F) steps (A) through (E) are reiterated on the next experimental space until data results selected in step (D) represent satisfactory leads.

In yet another embodiment, a system for investigating a catalyzed experimental space, comprises a reactor for effecting a CHTS method on the catalyzed chemical experimental space to produce results and a programmed controller to analyze the results according to matrix algebra to select a best case set of factor levels from the catalyzed experimental space.

### BRIEF DESCRIPTION OF THE DRAWINGS

FIG. 1 is a graphic representation of a complex catalyzed chemical experimental space;

FIG. 2 is a schematic representation of a CHTS system;

FIG. 3 shows representations of vector matrix forms; and

FIG. 4 is a normal probability plot.

### DETAILED DESCRIPTION OF THE INVENTION

A purpose of a CHTS experiment is to locate anomalies that represent high-value “leads.” Leads are results that identify candidate factors and levels for a commercial process. According to the invention, matrix algebra is combined with CHTS to examine the very high-level interactions of a chemical catalytic reaction. In the CHTS/matrix algebra approach, a CHTS experiment in many factors can be run and then modeled to generate all main, 2^{nd}, 3^{rd}, 4^{th} and even higher interactions. Suitably, the results can be represented according to a general linear model (GLM). A statistical analysis of the represented results determines high-order interaction leads.

In a typical CHTS method, a multiplicity of tagged reactants is subjected to an iteration of steps of (A) (i) simultaneously reacting the reactants, (ii) identifying a multiplicity of tagged products of the reaction and (B) evaluating the identified products after completion of a single or repeated iteration (A). A typical CHTS can utilize advanced automated, robotic, computerized and controlled loading, reacting and evaluating procedures.

The results from the CHTS experiment are analyzed using matrix algebra to extract combinations of the experiment interactions. A mathematical matrix is a representation of real numbers in a rectangular array. Matrices are important tools for expressing and discussing problems that may involve complex data sets. Matrix analysis is a multivariant methodology for expressing and manipulating these kinds of data and for solving problems posed by the data. Matrix operations can include representation (posing or modeling data in a matrix representation), addition, subtraction, scalar multiplication, matrix multiplication, multiplication by inverse, transposition (interchanging rows and columns) and distribution (assigning a probability value). Matrices can be manipulated to produce a sum, difference, scalar multiple, matrix multiple, product or transpose.

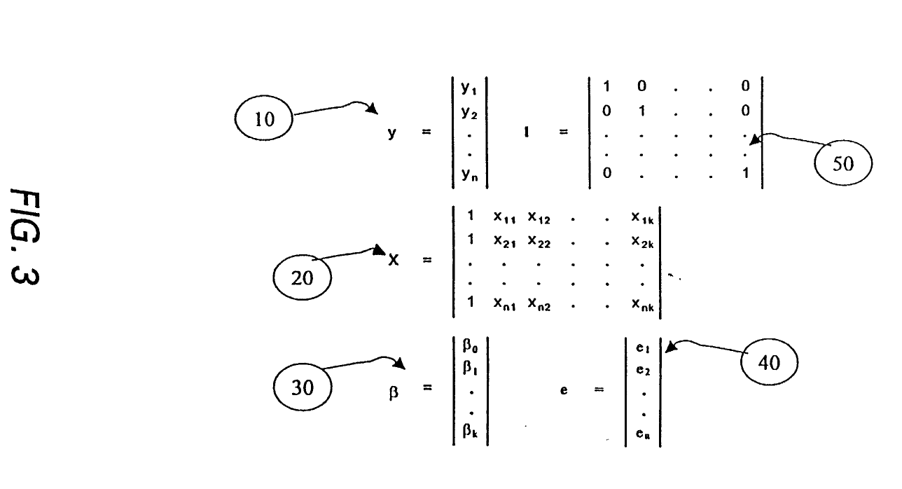

In the present invention, the matrices are representations of CHTS results in a rectangular array. The runs from the CHTS experiment provide sets of results or y's, one for each run, each correlated with a set of levels of factors, xₗ. Each run y is associated with an error e. Each of the factors or interactions is associated with a coefficient β. These elements (x's, their interactions and y's) can be represented in vector/matrix form as shown in FIG. 3, wherein levels of factors and interactions form a rectangular array or matrix (20) of scalar values X. Further in FIG. 3, y's, β's and e's are represented in single column matrices (10), (30), and (40). A matrix estimation equation of the system can be as follows:

y=Xβ+e (I)

where X is a matrix of factor and interaction levels in the experiment, y is a matrix of experimental results, β is effects (both main effects and interaction effects), every eᵢ in e has the same variance σ², error terms eᵢ arise from a normal distribution and expected value E(y) (definition: the value of the response if no error is present) of the response is E(y)=Xβ.

The method involves solving the above matrix estimation equation (I), according to the relationship:

β=(X′X)⁻¹X′y (II)

where superscript ′ indicates a transpose of a matrix (in which each row becomes a column and each column a row). The superscript ⁻¹ indicates an inverse function of a matrix. Thus for any square matrix A, a relation can be defined as AA⁻¹=I, where I is the identity matrix 50 shown in the FIG. 3 model.

Accordingly, results can be assembled as an n×1 vector y and factor level values can be assembled into an n×k+1 matrix X with 1's as designations in a first column and each other column containing the coded factor level values (+1's and −1's representing the extents of the values of the factors and interactions). Matrix equation (I) is then solved for effects parameters β.

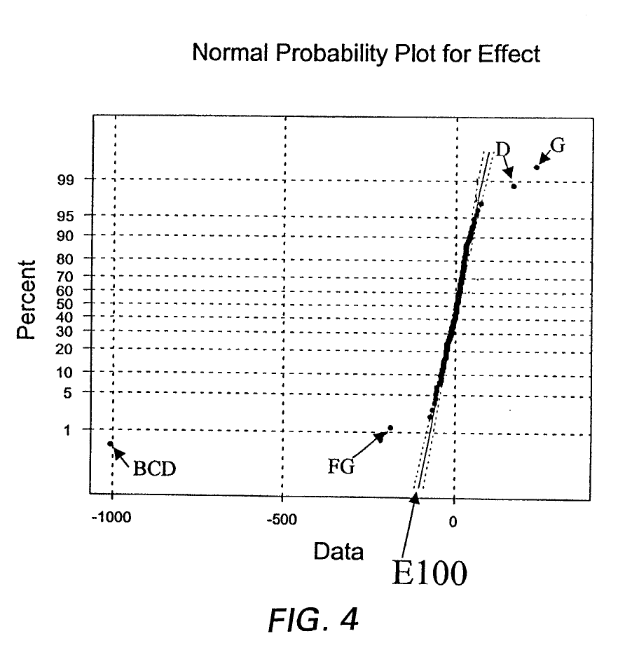

The effects parameters of matrix β are then examined for statistical significance. The null hypothesis can be applied in this examination. The null hypothesis is that all of the effects observed in the experiment are caused simply by random processes. If this is correct, the effects will fit to a normal distribution and form a relatively straight line in a probability plot. In FIG. 4; E100 is a straight line representing a results approximation. The line is flanked by dashed lines denoting multiples of standard deviations. A desired standard deviation can be selected by an experimenter for the experiment. Any effects that fall off the line by more than the standard deviation can be interpreted to have been caused by nonrandom processes, as taught by D. Montgomery, Design and Analysis of Experiments, 3^{rd} Ed., John Wiley, 1991, NY, p 99. Effects that positively exceed the deviation can represent combinations that are failures or combinations that provide synergistic improvement, i.e., leads.

In a preferred embodiment, results from the CHTS method are analyzed by matrix algebra by steps of (A) representing the results as an n×1 matrix y where n=a number of factor level combinations in the experiment, (B) representing extents of the factor level combinations in an n×n matrix X, (C) solving n simultaneous equations represented by the matrices according to matrix algebra to form a results matrix β and (D) examining the results matrix β to identify effects outside a standard deviation.

The step (B) can comprise coding extents of the factor level combinations as a+1 or −1 and representing the coded extents as the n×1 matrix y. The step (C) can comprise (i) transposing matrix X to form matrix X′, (ii) postmultiplying X′ by X to generate a matrix and (iii) postmultiplying the generated matrix by y to form the results matrix β. The step (D) can comprise (i) representing the results matrix β as a normal probability plot, defining a standard deviation for results of the plot and (iii) identifying positive interactions outside of the standard deviation. The standard deviation can represent a probability that a result deviation from the standard is random and that positive interactions can be identified outside of the deviation. In one embodiment, the probability can be established at 95 percent or better to define an experimental space for a commercial process or the probability can be established at 99.7 percent or better to define a best set of factor levels as leads for a commercial process.

In one embodiment, the invention is applied to screen for a catalyst to prepare a diaryl carbonate by carbonylation. Diaryl carbonates such as diphenyl carbonate can be prepared by reaction of hydroxyaromatic compounds such as phenol with oxygen and carbon monoxide in the presence of a catalyst composition comprising a Group VIIIB metal such as palladium or a compound thereof, a bromide source such as a quaternary ammonium or hexaalkylguanidinium bromide and a polyaniline in partially oxidized and partially reduced form.

Various methods for the preparation of diaryl carbonates by a carbonylation reaction of hydroxyaromatic compounds with carbon monoxide and oxygen have been disclosed. The carbonylation reaction requires a rather complex catalyst. Reference is made, for example, to Chaudhari et al., U.S. Pat. No. 5,917,077. The catalyst compositions described therein comprise a Group VIIIB metal (i.e., a metal selected from the group consisting of ruthenium, rhodium, palladium, osmium, iridium and platinum) or a complex thereof.

The catalyst material also includes a bromide source. This may be a quaternary ammonium or quaternary phosphonium bromide or a hexaalkylguanidinium bromide. The guanidinium salts are often preferred; they include the ∀, T-bis(pentaalkylguanidinium)alkane salts. Salts in which the alkyl groups contain 2-6 carbon atoms and especially tetra-n-butylammonium bromide and hexaethylguanidinium bromide are particularly preferred.

Other catalytic constituents are necessary in accordance with Chaudhari et al. The constituents include inorganic cocatalysts, typically complexes of cobalt(II) salts with organic compounds capable of forming complexes, especially pentadentate complexes. Illustrative organic compounds of this type are nitrogen-heterocyclic compounds including pyridines, bipyridines, terpyridines, quinolines, isoquinolines and biquinolines; aliphatic polyamines such as ethylenediamine and tetraalkylethylenediamines; crown ethers; aromatic or aliphatic amine ethers such as cryptanes; and Schiff bases. The especially preferred inorganic cocatalyst in many instances is a cobalt(II) complex with bis-3-(salicylalamino)propylmethylamine.

Organic cocatalysts may be present. These cocatalysts include various terpyridine, phenanthroline, quinoline and isoquinoline compounds including 2,2′:6′, 2″-terpyridine, 4-methylthio-2,2′:6′,2″-terpyridine and 2,2′:6′,2″-terpyridine N-oxide,1,10-phenanthroline, 2,4,7,8-tetramethyl-1,10-phenanthroline, 4,7-diphenyl-1,10, phenanthroline and 3,4,7,8-tetramethy-1,10-phenanthroline. The terpyridines and especially 2,2′:6′,2″-terpyridine are preferred.

Another catalyst constituent is a polyaniline in partially oxidized and partially reduced form.

Any hydroxyaromatic compound may be employed. Monohydroxyaromatic compounds, such as phenol, the cresols, the xylenols and p-cumylphenol are preferred with phenol being most preferred. The method may be employed with dihydroxyaromatic compounds such as resorcinol, hydroquinone and 2,2-bis(4-hydroxyphenyl)propane or “bisphenol A,” whereupon the products are polycarbonates.

Other reagents in the carbonylation process are oxygen and carbon monoxide, which react with the phenol to form the desired diaryl carbonate.

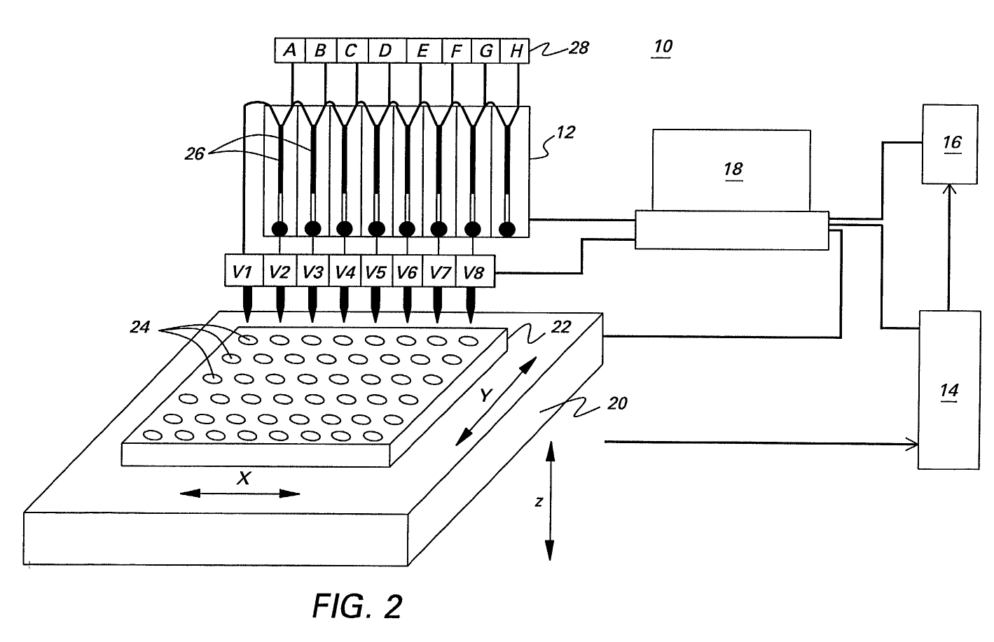

These and other features will become apparent from FIG. 2 and the following detailed discussion, which by way of example without limitation describe preferred embodiments of the present invention.

FIG. 2 is a schematic representation of a system 10 for CHTS according to the invention. FIG. 2 shows system 10 including dispensing assembly 12, reactor 14, detector 16 and controller 18. Further shown, is X-Y-Z robotic positioning stage 20, which supports array plate 22 with wells 24. The dispensing assembly 12 includes a battery of pipettes 26 that are controlled by controller 18. X-Y-Z robotic positioning stage 20 is controlled by controller 18 to position wells 24 of the array plate 22 beneath displacement pipettes 26 for delivery of test solutions from reservoirs 28.

Controller 18 controls aspiration of precursor solution into the battery of pipettes 26 and sequential positioning of the wells 24 of array plate 22 so that a prescribed stoichiometry and/or composition of reactant and/or catalyst can be delivered to the wells 24. By coordinating activation of the pipettes 26 and movement of plate 22 on the robotic X-Y-Z stage 20, a library of materials can be generated in a two-dimensional array for use in the CHTS method. Also, the controller 18 can be used to control sequence of charging of sample to reactor 14 and to control operation of the reactor 14 and the detector 16. Controller 18 can be a computer, processor, microprocessor or the like.

An experimental space definition defines the contents of the wells 24 for the CHTS method. The space can be defined according to any design that results in a representation of at least three factor interactions. Suitable designs include fractional factorial design, Latin square design, Plackett-Burman design or Taguchi design. Preferably the design results in a representation of all interactions and preferably, the design is an orthogonal design such as a full factorial design. The design can be embodied as an algorithm or program resident in controller 18.

Controller 18 controls the sequence of charging array plate 22 into the reactor 14, which is synchronized with operation of detector 16. Detector 16 detects products of reaction in the wells 24 of an array plate 22 after reaction in reactor 14. Detector 16 can utilize chromatography, infra red spectroscopy, mass spectroscopy, laser mass spectroscopy, microspectroscopy, NMR or the like to determine the constituency of each reaction product. The controller 18 uses data on the sample charged by the pipettes 26 and on the constituency of reaction product for each sample from detector 16 to correlate a detected product with at least one varying parameter of reaction. Additionally, an algorithm or program can be resident in the controller 18 to represent the CHTS results according to a matrix form and to analyze the represented results by matrix algebra to determine leads.

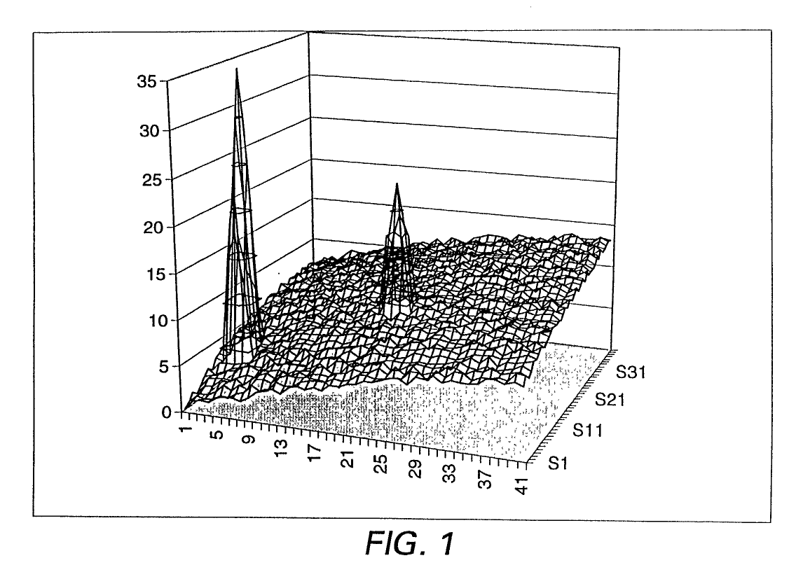

As an example, if the method and system of FIG. 1 is applied to study a carbonylation catalyst and/or to determine optimum carbonylation reaction conditions, the detector 16 analyzes the contents of the well for carbonylated product. In this case, the detector 16 can use Raman spectroscopy. The Raman peak is integrated using the analyzer electronics and the resulting data can be stored in the controller 18. Other analytical methods may be used—for example, Infrared spectrometry, mass spectrometry, headspace gas-liquid chromatography and fluorescence detection.

A method of screening complex catalyzed chemical reactions can be conducted in the FIG. 2 system 10. According to the method, catalyzed formulations are prepared according to any suitable procedure. For example, one procedure produces a homogeneous chemical reaction utilizing multiphase reactant systems. In this procedure, a formulation is prepared that represents a first reactant system that is at least partially embodied in a liquid. Each formulation is loaded as a thin film to a respective well 24 of the array plate 22 and the plate 22 is charged into reactor 14. During the subsequent reaction, the liquid of the first reactant system embodied is contacted with a second reactant system at least partially embodied in a gas. The liquid forms a film having a thickness sufficient to allow the reaction rate of the reaction to be essentially independent of the mass transfer rate of the second reactant system into the liquid.

The method herein described can be used with any suitable catalyzed chemical reactant system. For example, the system and method herein can be used for determining a method for producing diphenyl carbonate (DPC). Diphenyl carbonate (DPC) is useful, inter alia, as an intermediate in the preparation of polycarbonates. One method for producing DPC involves the carbonylation of a hydroxyaromatic compound (e.g., phenol) in the presence of a catalyst system. A carbonylation catalyst system typically includes a Group VIII B metal (e.g., palladium), a halide composition and a combination of inorganic co-catalysts (IOCCs).

Generally, testing of new catalyst systems has been accomplished at macro-scale and, because the mechanism of this carbonylation reaction is not fully understood, the identity of additional effective IOCCs has eluded practitioners. An embodiment of the present invention allows a homogeneous carbonylation reaction to be carried out in parallel with various potential catalyst systems and, consequently, this embodiment can be used to identify effective IOCCs for the carbonylation of phenol.

The following Example is illustrative and should not be construed as a limitation on the scope of the claims unless a limitation is specifically recited.

### EXAMPLE

This EXAMPLE illustrates an identification of an active and selective catalyst for the production of aromatic carbonates. The procedure identifies the best catalyst from a complex chemical space, where the chemical space is defined as an assemblage of all possible experimental conditions defined by a set of variable parameters such as formulation ingredient identity or amount or process parameter such as reaction time, temperature, or pressure.

The chemical space consists of the following TABLE 1 chemical factor levels and TABLE 2 processing factor levels:

TABLE 1

| Factor | Level | Level |
|---|---|---|
| Primary Catalyst | Ru(acac)3 | All at 25 ppm |
| | Pt(acac)2 | |
| Metal Cocatalyst | Mn(acac)2 | 150 and 1500 ppm |
| | Fe(acac)3 | |
| Cosolvent | Dimethylformamide | All at 10% |
| | (DMFA), Tetrahydrofuran | |
| | (THF) | |
| Anion Cocatalyst | Cl⁻, Br⁻, (as | All at 5000 ppm |
| | hexamethylguanadinium | |
| | salts) | |

TABLE 2

| Factor | Level |
|---|---|
| Pressure | 1000 psi, 1500 psi (8% Oxygen in Carbon Monoxide) |
| Temperature | 100 C., 120 C. |

The system has seven factors, each at two levels. There are 2⁷=128 possible combinations of these levels. The experiment is set up according to a full factorial design with 128 runs as shown in TABLE 3. In the experiment, catalyzed mixtures are made up in phenol solvent using the concentrations of each component as given in the rows of TABLE 3. The total volume of each catalyzed mixture is 1.0 ml. From each mixture, a 25 microliter aliquot is dispensed into a 2 ml reaction vial, forming a film on the bottom. The vials are grouped in array plates by process conditions (as specified in the Pressure and Temperature columns in the table) and each array plate is loaded into a high pressure autoclave and subjected to the reaction conditions specified. At the end of the reaction time, the reactor is cooled and depressurized and the contents of each vial are analyzed for diphenyl carbonate product using a gas chromatographic method. A turnover number (TON) for each reaction is calculated as mols of diphenylcarbonate/mols of primary catalyst. The results are given in the TON column of TABLE 3.

TABLE 3

| | B: | C: | | E: | | | |
|---|---|---|---|---|---|---|---|
| A: | Metal | Metal | D: | Anion | F: | | |
| Primary | Cocat- | Cocatalyst | Cosol- | Cocat- | Pres- | G: | |
| Catalyst | alyst | Amount | vent | alyst | sure | Temp. | TON |
| Pt | Mn | 150 | DMFA | Br | 1000 | 100 | 3340 |
| Ru | Mn | 150 | DMFA | Br | 1000 | 100 | 3470 |
| Pt | Fe | 150 | DMFA | Br | 1000 | 100 | 2360 |
| Ru | Fe | 150 | DMFA | Br | 1000 | 100 | 2260 |
| Pt | Mn | 1500 | DMFA | Br | 1000 | 100 | 2310 |
| Ru | Mn | 1500 | DMFA | Br | 1000 | 100 | 2060 |
| Pt | Fe | 1500 | DMFA | Br | 1000 | 100 | 3030 |
| Ru | Fe | 1500 | DMFA | Br | 1000 | 100 | 3200 |
| Pt | Mn | 150 | THF | Br | 1000 | 100 | 2430 |
| Ru | Mn | 150 | THF | Br | 1000 | 100 | 2270 |
| Pt | Fe | 150 | THF | Br | 1000 | 100 | 2910 |
| Ru | Fe | 150 | THF | Br | 1000 | 100 | 3160 |
| Pt | Mn | 1500 | THF | Br | 1000 | 100 | 3270 |
| Ru | Mn | 1500 | THF | Br | 1000 | 100 | 3030 |
| Pt | Fe | 1500 | THF | Br | 1000 | 100 | 2260 |
| Ru | Fe | 1500 | THF | Br | 1000 | 100 | 2470 |
| Pt | Mn | 150 | DMFA | Cl | 1000 | 100 | 3040 |
| Ru | Mn | 150 | DMFA | Cl | 1000 | 100 | 3340 |
| Pt | Fe | 150 | DMFA | Cl | 1000 | 100 | 2030 |
| Ru | Fe | 150 | DMFA | Cl | 1000 | 100 | 1860 |
| Pt | Mn | 1500 | DMFA | Cl | 1000 | 100 | 2200 |
| Ru | Mn | 1500 | DMFA | Cl | 1000 | 100 | 1920 |
| Pt | Fe | 1500 | DMFA | Cl | 1000 | 100 | 3290 |
| Ru | Fe | 1500 | DMFA | Cl | 1000 | 100 | 2910 |
| Pt | Mn | 150 | THF | Cl | 1000 | 100 | 2260 |
| Ru | Mn | 150 | THF | Cl | 1000 | 100 | 2410 |
| Pt | Fe | 150 | THF | Cl | 1000 | 100 | 3260 |
| Ru | Fe | 150 | THF | Cl | 1000 | 100 | 3200 |
| Pt | Mn | 1500 | THF | Cl | 1000 | 100 | 3360 |
| Ru | Mn | 1500 | THF | Cl | 1000 | 100 | 3090 |
| Pt | Fe | 1500 | THF | Cl | 1000 | 100 | 2320 |
| Ru | Fe | 1500 | THF | Cl | 1000 | 100 | 2320 |
| Pt | Mn | 150 | DMFA | Br | 1500 | 100 | 3230 |
| Ru | Mn | 150 | DMFA | Br | 1500 | 100 | 3710 |
| Pt | Fe | 150 | DMFA | Br | 1500 | 100 | 2140 |
| Ru | Fe | 150 | DMFA | Br | 1500 | 100 | 2500 |
| Pt | Mn | 1500 | DMFA | Br | 1500 | 100 | 2490 |
| Ru | Mn | 1500 | DMFA | Br | 1500 | 100 | 2230 |
| Pt | Fe | 1500 | DMFA | Br | 1500 | 100 | 3070 |
| Ru | Fe | 1500 | DMFA | Br | 1500 | 100 | 3360 |
| Pt | Mn | 150 | THF | Br | 1500 | 100 | 2680 |
| Ru | Mn | 150 | THF | Br | 1500 | 100 | 2640 |
| Pt | Fe | 150 | THF | Br | 1500 | 100 | 3520 |
| Ru | Fe | 150 | THF | Br | 1500 | 100 | 3620 |
| Pt | Mn | 1500 | THF | Br | 1500 | 100 | 3620 |
| Ru | Mn | 1500 | THF | Br | 1500 | 100 | 3830 |
| Pt | Fe | 1500 | THF | Br | 1500 | 100 | 2710 |
| Ru | Fe | 1500 | THF | Br | 1500 | 100 | 2580 |
| Pt | Mn | 150 | DMFA | Cl | 1500 | 100 | 3310 |
| Ru | Mn | 150 | DMFA | Cl | 1500 | 100 | 3740 |
| Pt | Fe | 150 | DMFA | Cl | 1500 | 100 | 2460 |
| Ru | Fe | 150 | DMFA | Cl | 1500 | 100 | 2410 |
| Pt | Mn | 1500 | DMFA | Cl | 1500 | 100 | 2020 |
| Ru | Mn | 1500 | DMFA | Cl | 1500 | 100 | 2300 |
| Pt | Fe | 1500 | DMFA | CI | 1500 | 100 | 3270 |
| Ru | Fe | 1500 | DMFA | Cl | 1500 | 100 | 3250 |
| Pt | Mn | 150 | THF | Cl | 1500 | 100 | 2800 |
| Ru | Mn | 150 | THF | Cl | 1500 | 100 | 2520 |
| Pt | Fe | 150 | THF | Cl | 1500 | 100 | 3850 |
| Ru | Fe | 150 | THF | Cl | 1500 | 100 | 3570 |
| Pt | Mn | 1500 | THF | Cl | 1500 | 100 | 3540 |
| Ru | Mn | 1500 | THF | Cl | 1500 | 100 | 3950 |
| Pt | Fe | 1500 | THF | Cl | 1500 | 100 | 2730 |
| Ru | Fe | 1500 | THF | Cl | 1500 | 100 | 2520 |
| Pt | Mn | 150 | DMFA | Br | 1000 | 120 | 3300 |
| Ru | Mn | 150 | DMFA | Br | 1000 | 120 | 3800 |
| Pt | Fe | 150 | DMFA | Br | 1000 | 120 | 2640 |
| Ru | Fe | 150 | DMFA | Br | 1000 | 120 | 2520 |
| Pt | Mn | 1500 | DMFA | Br | 1000 | 120 | 2220 |
| Ru | Mn | 1500 | DMFA | Br | 1000 | 120 | 2270 |
| Pt | Fe | 1500 | DMFA | Br | 1000 | 120 | 3960 |
| Ru | Fe | 1500 | DMFA | Br | 1000 | 120 | 3360 |
| Pt | Mn | 150 | THF | Br | 1000 | 120 | 2560 |
| Ru | Mn | 150 | THF | Br | 1000 | 120 | 2440 |
| Pt | Fe | 150 | THF | Br | 1000 | 120 | 3560 |
| Ru | Fe | 150 | THF | Br | 1000 | 120 | 3620 |
| Pt | Mn | 1500 | THF | Br | 1000 | 120 | 3650 |
| Ru | Mn | 1500 | THF | Br | 1000 | 120 | 3570 |
| Pt | Fe | 1500 | THF | Br | 1000 | 120 | 2650 |
| Ru | Fe | 1500 | THF | Br | 1000 | 120 | 2710 |
| Pt | Mn | 150 | DMFA | Cl | 1000 | 120 | 3380 |
| Ru | Mn | 150 | DMFA | Cl | 1000 | 120 | 3430 |
| Pt | Fe | 150 | DMFA | Cl | 1000 | 120 | 2480 |
| Ru | Fe | 150 | DMFA | Cl | 1000 | 120 | 2310 |
| Pt | Mn | 1500 | DMFA | Cl | 1000 | 120 | 2510 |
| Ru | Mn | 1500 | DMFA | Cl | 1000 | 120 | 2410 |
| Pt | Fe | 1500 | DMFA | Cl | 1000 | 120 | 3320 |
| Ru | Fe | 1500 | DMFA | Cl | 1000 | 120 | 3620 |
| Pt | Mn | 150 | THF | CI | 1000 | 120 | 2880 |
| Ru | Mn | 150 | THF | Cl | 1000 | 120 | 2430 |
| Pt | Fe | 150 | THF | Cl | 1000 | 120 | 3940 |
| Ru | Fe | 150 | THF | Cl | 1000 | 120 | 4290 |
| Pt | Mn | 1500 | THF | Cl | 1000 | 120 | 3730 |
| Ru | Mn | 1500 | THF | Cl | 1000 | 120 | 3730 |
| Pt | Fe | 1500 | THF | Cl | 1000 | 120 | 2730 |
| Ru | Fe | 1500 | THF | Cl | 1000 | 120 | 2480 |
| Pt | Mn | 150 | DMFA | Br | 1500 | 120 | 3510 |
| Ru | Mn | 150 | DMFA | Br | 1500 | 120 | 3420 |
| Pt | Fe | 150 | DMFA | Br | 1500 | 120 | 2340 |
| Ru | Fe | 150 | DMFA | Br | 1500 | 120 | 2390 |
| Pt | Mn | 1500 | DMFA | Br | 1500 | 120 | 2210 |
| Ru | Mn | 1500 | DMFA | Br | 1500 | 120 | 2660 |
| Pt | Fe | 1500 | DMFA | Br | 1500 | 120 | 3270 |
| Ru | Fe | 1500 | DMFA | Br | 1500 | 120 | 3550 |
| Pt | Mn | 150 | THF | Br | 1500 | 120 | 2450 |
| Ru | Mn | 150 | THF | Br | 1500 | 120 | 2830 |
| Pt | Fe | 150 | THF | Br | 1500 | 120 | 3910 |
| Ru | Fe | 150 | THF | Br | 1500 | 120 | 3850 |
| Pt | Mn | 1500 | THF | Br | 1500 | 120 | 3470 |
| Ru | Mn | 1500 | THF | Br | 1500 | 120 | 3730 |
| Pt | Fe | 1500 | THF | Br | 1500 | 120 | 2360 |
| Ru | Fe | 1500 | THF | Br | 1500 | 120 | 2660 |
| Pt | Mn | 150 | DMFA | Cl | 1500 | 120 | 3640 |
| Ru | Mn | 150 | DMFA | CL | 1500 | 120 | 3440 |
| Pt | Fe | 150 | DMFA | Cl | 1500 | 120 | 2380 |
| Ru | Fe | 150 | DMFA | Cl | 1500 | 120 | 2230 |
| Pt | Mn | 1500 | DMFA | Cl | 1500 | 120 | 2510 |
| Ru | Mn | 1500 | DMFA | Cl | 1500 | 120 | 2150 |
| Pt | Fe | 1500 | DMFA | Cl | 1500 | 120 | 3100 |
| Ru | Fe | 1500 | DMIFA | Cl | 1500 | 120 | 3810 |
| Pt | Mn | 150 | THF | Cl | 1500 | 120 | 2380 |
| Ru | Mn | 150 | THF | Cl | 1500 | 120 | 2900 |
| Pt | Fe | 150 | THF | Cl | 1500 | 120 | 3740 |
| Ru | Fe | 150 | THF | Cl | 1500 | 120 | 3620 |
| Pt | Mn | 1500 | THF | Cl | 1500 | 120 | 3610 |
| Ru | Mn | 1500 | THF | Cl | 1500 | 120 | 3680 |
| Pt | Fe | 1500 | THF | Cl | 1500 | 120 | 2570 |
| Ru | Fe | 1500 | THF | Cl | 1500 | 120 | 2880 |

Analysis of the data using matrix estimation formula (I) gives the information of TABLE 4. The Terms are the main effects and interactions of the factors in TABLE 3 and A-F are as given in the heading of TABLE 3. Thus A is the main effect of Factor “Primary Catalyst” and AD is the interaction effect of the Factors “Primary Catalyst” and “Cosolvent.” Effects are β's.

TABLE 4

| Term | Effect (β) | Term | Effect (β) | Term | Effect (β) |
|---|---|---|---|---|---|
| A | −25 | BFG | 22 | ABDEF | −24 |
| B | 2 | CDE | −24 | ABDEG | −7 |
| C | −43 | CDF | −14 | ABDFG | −58 |
| D | 164 | CDG | 1 | ABEFG | 9 |
| E | 32 | CEF | −67 | ACDEF | −25 |
| F | 20 | CEG | 20 | ACDEG | 22 |
| G | 229 | CFG | 13 | ACDFG | −16 |
| AB | 10 | DEF | 1 | ACEFG | 51 |
| AC | 2 | DEG | −16 | ADEFG | −6 |
| AD | 7 | DFG | 40 | BCDEF | 3 |
| AE | 22 | EFG | −37 | BCDEG | 21 |
| AF | −26 | ABCD | 2 | BCDFG | −40 |
| AG | 42 | ABCE | −52 | BCEFG | 9 |
| BC | 39 | ABCF | −40 | BDEFG | −26 |
| BD | 29 | ABCG | 30 | CDEFG | 18 |
| BE | 73 | ABDE | 44 | ABCDEF | 51 |
| BF | 16 | ABDF | 13 | ABCDEG | 1 |
| BG | −12 | ABDG | 21 | ABCDFG | 20 |
| CD | 6 | ABEF | −11 | ABCEFG | −73 |
| CE | −28 | ABEG | 13 | ABDEFG | −57 |
| CF | 23 | ABFG | 20 | ACDEFG | −4 |
| CG | −27 | ACDE | −4 | BCDEFG | 4 |
| DE | 28 | ACDF | −22 | ABCDEFG | −11 |
| DF | −31 | ACDG | 12 | | |
| DG | −35 | ACEF | −40 | | |
| EF | −9 | ACEG | 21 | | |
| EG | −25 | ACFG | −32 | | |
| FG | −188 | ADEF | 7 | | |
| ABC | −21 | ADEG | −41 | | |
| ABD | 13 | ADFG | −1 | | |
| ABE | −6 | AEFG | 4 | | |
| ABF | 4 | BCDE | −56 | | |
| ABG | 1 | BCDF | 14 | | |
| ACD | −14 | BCDG | 24 | | |
| ACE | 1 | BCEF | −8 | | |
| ACF | 27 | BCEG | 29 | | |
| ACG | −20 | BCFG | 6 | | |
| ADE | 30 | BDEF | 6 | | |
| ADF | −17 | BDEG | 12 | | |
| ADG | −6 | BDFG | −32 | | |
| AEF | −53 | BEFG | 30 | | |
| AEG | 17 | CDEF | 19 | | |
| AFG | 53 | CDEG | 18 | | |
| BCD | −1005 | CDFG | 7 | | |
| BCE | −36 | CEFG | 58 | | |
| BCF | 14 | DEFG | −2 | | |
| BCG | 28 | ABCDE | −8 | | |
| BDE | −10 | ABCDF | 16 | | |
| BDF | 47 | ABCDG | 29 | | |
| BDG | 7 | ABCEF | −23 | | |
| BEF | 36 | ABCEG | −34 | | |

The Effects are fitted to a normal probability plot and four points are identified as falling outside two standard deviations of the straight line fit: D, G, FG, and BCD. The BCD interaction is identified as a potential lead to nonlinear behavior simultaneously involving the Cocatalyst Metal (B), the Cocatalyst Metal Amount (C), and the Cosolvent (D). Repeated followup iterations identify a strong synergistic effect of high levels of Fe in the presence of THF.

While preferred embodiments of the invention have been described, the present invention is capable of variation and modification and therefore should not be limited to the precise details of the EXAMPLE. The invention includes changes and alterations that fall within the purview of the following claims.

What is claimed is:

1. A method, comprising:
defining an experimental space of a catalyzed chemical reaction to represent at least three factor interactions,
effecting a combinatorial high throughput screening (CHTS) method on the catalyzed chemical experimental space to produce results; and
analyzing the results according to matrix algebra to select a best case set of factor levels from the catalyzed experimental space.

2. The method of claim 1, wherein the experimental space is defined to represent all interactions of factors of the reaction.

3. The method of claim 1, wherein the experimental space is defined according to a full factorial design.

4. The method of claim 1, wherein the results from the matrix algebra analysis are represented according to a general linear model.

5. The method of claim 1, wherein the experimental space is defined according to a full factorial design that represents at least 6 orders of interaction of factors of the reaction.

6. The method of claim 1, wherein the experimental space is defined according to a full factorial design that represents at least 9 orders of interaction of factors of the reaction.

7. The method of claim 1, wherein the experimental space is defined according to a full factorial design that represents all orders of interaction of factors of the reaction.

8. The method of claim 1, wherein the analyzing step comprises:
(A) representing the results as an n×1 matrix y where n=a number of factor level combinations in the experiment;
(B) representing extents of the factor level combinations in an n×n matrix X;
(C) solving n simultaneous equations represented by the matrices according to matrix algebra to form a results matrix β; and
(D) examining the results matrix β to identify effects outside a standard deviation.

9. The method of claim 8, wherein (B) comprises coding extents of the factor level combinations as a +1 or −1 and representing the coded extents as the n×1 matrix y.

10. The method of claim 8, wherein (C) comprises:
(i) transposing matrix X to form matrix X′;
(ii) postmultiplying X′ by X to generate a matrix; and
(iii) postmultiplying the generated matrix by y to form the results matrix β.

11. The method of claim 8, wherein (D) comprises:
(i) representing the results matrix β as a normal probability plot;
(ii) defining a standard deviation for results of the plot; and
(iii) identifying positive interactions outside of the standard deviation.

12. The method of claim 11, wherein the standard deviation represents a probability that a result deviation from the standard is random and that a positive interaction can be identified outside of the deviation.

13. The method of claim 12, wherein the probability is established at 95 percent or better.

14. The method of claim 12, wherein the probability is established at 99.7 percent or better.

15. The method of claim 11, wherein the positive interactions are results that represent a best set of factor levels from the experimental space.

16. The method of claim 15, wherein the best set of factor levels defines leads for a commercial process.

17. The method of claim 15, wherein the best set of factor levels defines a space for further investigation by reiteration of a CHTS method.

18. The method of claim 1, wherein the matrix algebra analysis comprises representing the results according to the following model equation (I)
y=Xβ+e (I)
where X is a matrix of factor and interaction levels in the experiment, y is a matrix of experimental results, β is effects and e is an error term of variance σ² from a normal distribution.

19. The method of claim 18, wherein the matrix algebra analysis comprises assembling results as an n×1 vector y, assembling factor level values into an n×k+1 matrix X, representing extents of the results and factor level values as +1's and −1's accordingly and solving for effects parameters β according to the relationship:
β=(i X′X)⁻¹X′y (II)
where superscript ′ is a transpose of a matrix and superscript ⁻¹ identifies an inverse function of a matrix.

20. The method of claim 19, comprising examining the solved effects parameters β to identify effects outside a standard deviation.

21. The method of claim 20, further comprising reiterating the CHTS method wherein an experimental space for the CHTS method is selected according to the identified effects.

22. The method of claim 1, further comprising applying a statistical analysis to the results to identify interactions that represent a best set of factor levels from the experimental space.

23. The method of claim 1, wherein the CHTS comprises effecting parallel chemical reactions of an array of reactants defined as the experimental space.

24. The method of claim 1, wherein the CHTS comprises effecting parallel chemical reactions on a micro scale on reactants defined as the experimental space.

25. The method of claim 1, wherein the CHTS comprises an iteration of steps of simultaneously reacting a multiplicity of tagged reactants and identifying a multiplicity of tagged products of the reaction and evaluating the identified products after completion of a single or repeated iteration.

26. The method of claim 1, wherein the experimental space factors comprise reactants, catalysts and conditions and the CHTS comprises
(A) (a) reacting a reactant selected from the experimental space under a selected set of catalysts or reaction conditions; and (b) evaluating a set of results of the reacting step; and
(B) reiterating step (A) wherein a selected experimental space selected for a step (a) is chosen as a result of an evaluating step (b) of a preceding iteration of step (A).

27. The method of claim 26, wherein the evaluating step (b) comprises identifying relationships between factor levels of the candidate chemical reaction space; and determining the chemical experimental space according to a full factorial design for the next iteration.

28. The method of claim 26, comprising reiterating (A) until a best set of factor levels of the chemical experimental space is selected.

29. The method of claim 1, wherein the chemical space includes a catalyst system comprising a Group VIII B metal.

30. The method of claim 1, wherein the chemical space includes a catalyst system comprising palladium.

31. The method of claim 1, wherein the chemical space includes a catalyst system comprising a halide composition.

32. The method of claim 1, wherein the chemical space includes an inorganic co-catalyst.

33. The method of claim 1, wherein the chemical space includes a catalyst system includes a combination of inorganic co-catalysts.

34. The method of claim 1, wherein the defined space comprises a reactant or catalyst at least partially embodied in a liquid and effecting the CHTS method comprises contacting the reactant or catalyst with an additional reactant at least partially embodied in a gas, wherein the liquid forms a film having a thickness sufficient to allow a reaction rate that is essentially independent of a mass transfer rate of additional reactant into the liquid to synthesize products that comprise the results.

35. A method of conducting an experiment, comprising steps of:
(A) conducting a CHTS experiment on a complex experimental space comprising qualitative and quantitative factors to produce first data results;
(B) analyzing the first data results according to matrix algebra;
(C) defining a standard deviation of the analyzed results;
(D) selecting data results that positively exceed the standard deviation,
(E) defining a next experimental space according to the selected data results; and
(F) reiterating steps (A) through (E) on the next experimental space until data results selected in step (D) represent satisfactory leads.

36. A system for investigating a catalyzed experimental space, comprising;
a reactor for effecting a CHTS method on the catalyzed chemical experimental space to produce results; and
a programmed controller that analyzes the results according to matrix algebra to select a best case set of factor levels from the catalyzed experimental space.

37. The system of claim 36, comprising a programmed controller that analyzes the results according to matrix algebra and represents the results of the analysis according to a substantially linear model.

38. The system of claim 36, comprising a programmed controller to define the catalyzed chemical experimental space to represent at least three factor interactions.

39. The system of claim 36, wherein the controller is a computer, processor or microprocessor.

40. The system of claim 36, further comprising a dispensing assembly to charge factor levels of reactants or catalysts representing the catalyzed chemical experimental space to wells of an array plate for charging to the reactor.

41. The system of claim 39, comprising a programmed controller to define the catalyzed chemical experimental space and to control the assembly to charge factor levels of reactants or catalysts according to the controller defined space.

42. The system of claim 36, further comprising a detector to detect results of the CHTS method effected in the reactor.
